(19) United States
(12) Patent Application Publication
(10) Pub. No.: US 2009/0297570 A1
(43) Pub. Date: Dec. 3, 2009

(54) ORAL CARE COMPOSITION

(76) Inventor: Brian Joseph Groves, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/309,891

(22) PCT Filed: Jul. 30, 2007
(86) PCT No.: PCT/EP2007/057829

§ 371 Date: Aug. 13, 2009

(30) Foreign Application Priority Data
Aug. 3, 2006 (EP) 06118389.3

(51) Int. Cl. A61K 8/02; A61K 8/92; A61Q 11/00; B01J 13/22

(52) U.S. Cl. 424/401; 424/49; 264/4.4

(57) ABSTRACT

An oral care composition comprising from 0.01 to 50% by weight of the composition of a capsule, the capsule comprising a core and a coating for the core, the coating comprising a wax having a melting point of at least 70 degrees C., an oil and an opacifier and the core comprising an oil, a pigment and a shear-thinning structurant.

The present invention relates to an oral care composition comprising a pigment and also to a method for manufacturing capsules comprising a pigment.

WO 2005/123023 (Ribi) discloses a toothpaste composition comprising a material which is capable of changing colour on one pH change.

In a first aspect the present invention there is provided an oral care composition comprising from 0.01 to 50% by weight of the composition of a capsule, the capsule comprising a core and a coating for the core, the coating comprising a wax having a melting point of at least 70 degrees C., an oil and an opacifier and the core comprising an oil, a pigment and a shear-thinning structurant. Such a composition provides a useful indication to the user that toothbrushing has occurred for a predetermined time, for example, the professionally recommended two minutes.

In a second aspect of the present invention, there is provided a method of manufacturing capsules comprising pigment, said method comprising dispersing a pigment in oil and adding a shear-thinning structurant to gel the dispersion; heating an oil, a wax of melting point of at least 70 degrees C., and an opacifier to form a free flowing dispersion of the opacifier in molten wax/oil; and encapsulating the gelled dispersion of pigment in oil within the dispersion of opacifier in molten wax/oil.

The nature of the capsules used in the present invention enables good containment of pigment within the capsule until shear caused during toothbrushing leads to the breakdown of the capsules and the release of the pigment. For the capsules to function in the correct manner, showing colour only after the desired time, both the nature of the coating and the nature of the core of the capsules are important. Thus, it is important that the coating comprises a wax as described herein, an oil, and an opacifier and it is important that the core comprises an oil, a pigment, and a shear-thinning structurant.

The capsules are present in an amount ranging from 0.01 to 50% by weight of the composition. Preferably, they comprise from 0.5 to 10% by weight of the composition. Such an amount provides the optimum sensory during use and also the optimum pigment release profile when the capsules are broken during toothbrushing.

Typically, the coating of the capsules comprises from 20 to 60% of the total weight of the capsules, preferably from 30 to 50%, and more preferably around 40%.

The wax present in the coating is preferably one with a melting point of greater than 70 degrees C., more preferably greater than 80 degrees C. Such waxes include carnauba wax, Chinese insect wax, candililla wax, castor wax, Japan wax, shellac, ouricouri and synthetic waxes such as high molecular weight paraffin wax and microcrystalline wax.

The capsule coating preferably comprises from 20 to 60% by weight of wax with a melting point of greater than 70 degrees C. and more preferably from 30 to 50%.

The oil, both in the coating and in the core, is preferably obtained from an animal, vegetable or mineral source. Preferred sources include vegetable sources such as fruits and plant seeds. Preferred oils include olive oil, borage oil, primrose oil, groundnut oil, canola oil, safflower oil, rice bran oil, peanut oil and other plant seed oils including sunflower (seed) oil. The most preferred sunflower oil is the so-called high-oleate sunflower oil which presents a higher resistance to rancidity.

The oil in the coating may be different to the oil in the core.

The capsule coating preferably comprises from 35 to 75% by weight of oil and more preferably from 50 to 65%.

The preferred ratio between the wax and the oil in the coating is from 20:80 to 80:20, more preferably from 30:70 to 40:60. These ratios provide the capsule with the best resistance to fracture during processing and also the best pigment release profile.

Preferably the opacifier is present at an amount to mask any colour in the core, preferably this is from 1 to 10% by weight of the coating, more preferably from 3 to 7% by weight of the coating. Preferably the opacifier is titanium dioxide.

Typically, the core comprises from 40 to 80% by weight of the capsule, preferably from 50 to 70% and more preferably around 60% by weight of the capsule.

The oil is present at from 50 to 95% by weight of the core, preferably from 60 to 90 and more preferably from 70 to 80% by weight of the core.

The core also comprises a pigment. The preferred pigments are blue, red, green, yellow, purple or orange. The most preferred are blue. Especially preferred are those selected from CI 14720, CI 15985, CI 16035, CI 16255, CI 19140, CI 42051, CI 42053, CI 42090, CI 47005, CI 73360, CI 74160, CI 74260, CI 75470, CI 75810, CI 77007, CI 77491 and CI 77492. These pigments present the best sensory profile during toothbrushing.

The pigment is present at from 1 to 50% by weight of the core, preferably from 10 to 30% by weight of the core.

The shear-thinning structurant present in the core of the capsules is important in achieving the desired delayed release of pigment. The presence of the shear-thinning structurant also assists the manufacture of the capsules used in the present invention.

The shear-thinning structurant is present in the core at from 1 to 40% by weight of the core, preferably from 2 to 10 and more preferably from 4 to 6% by weight of the core.

A preferred shear-thinning structurant is fumed silica. The capsules preferably have a weight average particle size of from 350 to 800 μm, more preferably from 400 to 600 μm. These present the best sensory to the user during toothbrushing.

The capsules according to the invention provide the best pigment loading without compromising capsule integrity since the highly viscous interior core is unlikely to leak out of a capsule when the coating is breached. The pigment will only leak out when the capsule is completely ruptured, i.e. during brushing.

The oral composition preferably comprises not more than 0.3% by weight pigment based on its powder weight, preferably not more than 0.2% by weight.

The oral composition according to the invention may comprise further ingredients which are common in the art, such as:

antimicrobial agents, e.g. Triclosan, chlorhexidine, copper, zinc, and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);
anti-inflammatory agents such as ibuprofen, flurbiprofen, aspirin, indomethacin etc.;
anti-caries agents such as sodium- and stannous fluoride, aminefluorides, sodium monofluorophosphate, sodium trimeta phosphate and casein;
plaque buffers such as urea, calcium lactate, calcium glycerophosphate and strontium polyacrylates;
vitamins such as Vitamins A, C and E;
plant extracts;
desensitising agents, e.g. potassium citrate, potassium chloride, potassium tartrate, potassium bicarbonate, potassium oxalate, potassium nitrate and strontium salts;
anti-calculus agents, e.g. alkali-metal pyrophosphates, hypophosphite-containing polymers, organic phosphonates and phosphocitrates etc.;
biomolecules, e.g. bacteriocins, antibodies, enzymes, etc.;
flavours, e.g. peppermint and spearmint oils;
proteinaceous materials such as collagen;
preservatives;
opacifying agents;
colouring agents;
pH-adjusting agents;
sweetening agents;
pharmaceutically acceptable carriers, e.g. starch, sucrose, water or water/alcohol systems etc.;
surfactants, such as anionic, nonionic, cationic and zwitterionic or amphoteric surfactants;
particulate abrasive materials such as silicas, aluminas, calcium carbonates, dicalciumphosphates, calcium pyrophosphates, hydroxyapatites, trimetaphosphates, insoluble hexametaphosphates and so on, including capsuled particulate abrasive materials, usually in amounts between 3 and 60% by weight of the oral care composition.
humectants such as glycerol, sorbitol, propyleneglycol, xylitol, lactitol etc.;
binders and thickeners such as sodium carboxymethyl-cellulose, xanthan gum, gum arabic etc. as well as synthetic polymers such as polyacrylates and carboxyvinyl polymers such as Carbopol®;
polymeric compounds which can enhance the delivery of active ingredients such as antimicrobial agents can also be included. Examples of such polymers are copolymers of polyvinylmethylether with maleic anhydride and other similar delivery enhancing polymers, e.g. those described in DE-A-3,942,643 (Colgate);
buffers and salts to buffer the pH and ionic strength of the oral care composition; and other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

### EXAMPLES

Table 1 indicates the formulations of capsules as used in accordance with the present invention. Amounts of components are percentages by weight and the ratios of core:coating indicated are also by weight.

TABLE 1

Example

| | 1 | 2 | 3 | 4 |
|---|---|---|---|---|
| Core: | | | | |
| Olive oil | — | — | 72.5 | — |
| Sunflower oil | 75 | 70 | — | 80 |
| Pigment | 20 | 24 | 23.5 | 14 |
| Fumed silica | 5 | 6 | 4 | 6 |
| Coating: | | | | |
| Castor wax | — | — | 40 | — |
| Carnauba wax | 38.4 | 32 | — | 45 |
| Sunflower oil | 57.6 | 65 | 53 | 50 |
| Titanium | 4 | 3 | 7 | 5 |
| dioxide | | | | |
| Core:coating | 50:50 | 40:60 | 70:30 | 60:40 |

The following process was used to make the capsules of Example 1.

Pigment (10 g) was dispersed in sunflower oil (28.8 g) using an overhead stirrer. Hydrophobic fumed silica (2.5 g) was then added to the dispersion in order to gel it.

In a separate vessel, carnauba wax (19.2 g) was heated to 85-90° C. in order to melt it and sunflower oil (28.8 g) and titanium dioxide (2 g) were then mixed in using an overhead stirrer.

The gelled dispersion of pigment and sunflower oil was stirred into water (4 L) held at 85-90° C. To this mixture was added the hot carnauba wax, sunflower oil, titanium dioxide mixture, with stirring. When this addition was complete, the aqueous suspension of encapsulates was rapidly cooled to below the melting point of the carnauba wax, the encapsulates were extracted, and dried.

In an alternative process of manufacture, applicable to all co-extrusion encapsulation is used. In this process, there are two hot feeds (jacketed lines or electrical heat traced) that feed, via controllable pumps, to a co-extrusion nozzle consisting of an internal tube carrying core material and an external tube carrying the coating material. The nozzle generally has a heating means in order to prevent solidification of the molten wax in the coating.

To control the particle size of the capsules, a means for breaking up the dual stream of hot materials exiting the co-extrusion nozzle is employed. Typical means are vibratory or use pressurised, heated air.

To solidify the encapsulates, a cooling means is employed. A typical means is a cooling tunnel, although a cooled water “collection bath” may alternatively or additionally be employed.

1. An oral care composition comprising from 0.01 to 50% by weight of the composition of a capsule, the capsule comprising a core and a coating for the core, the coating comprising a wax having a melting point of at least 70 degrees C., an oil and an opacifier and the core comprising an oil, a pigment and a shear-thinning structurant.

2. An oral care composition according to claim 1 wherein the wax has a melting point of at least 80 degrees C.

3. An oral care composition according to claim 1 wherein the oil is sunflower oil.

4. An oral care composition according to claim 1 wherein the oil is high-oleate sunflower oil.

5. An oral care composition according to claim 1 wherein the wax is carnauba wax.

6. An oral care composition according to claim 1 wherein the weight ratio between the wax and the oil in the coating is from 20:80 to 80:20, preferably from 30:70 to 40:60.

7. An oral care composition according to claim 1 wherein the opacifier is titanium dioxide.

8. An oral care composition according to claim 1 wherein the shear thinning structurant is fumed silica.

9. An oral care composition according to claim 1 wherein the pigment is selected from—CI 14720, CI 15985, CI 16035, CI 16255, CI 19140, CI 42051, CI 42053, CI 42090, CI 47005, CI 73360, CI 74160, CI 74260, CI 75470, CI 75810, CI 77007, CI 77491 and CI 77492.

10. An oral care composition according to claim 1 wherein the weight average particle size of the capsules is from 350 to 800 μm, preferably from 400 to 600 μm.

11. An oral care composition according to claim 1 wherein the total amount of pigment is not greater than 0.3% by weight, preferably not more than 0.2% by weight.

12. An oral care composition according to claim 1 wherein the composition comprises a first phase and a second phase and wherein the capsule is present in only the first phase.

13. A method of manufacturing capsules comprising pigment, said method comprising dispersing a pigment in oil and adding a shear-thinning structurant to gel the dispersion; heating an oil, a wax of melting point of at least 70 degrees C., and an opacifier to form a free flowing dispersion of the opacifier in molten wax/oil; and encapsulating the gelled dispersion of pigment in oil within the dispersion of opacifier in molten wax/oil.
